(19) United States
(12) Patent Application Publication
(10) Pub. No.: US 2020/0368489 A1
(43) Pub. Date: Nov. 26, 2020

(54) MINDFULNESS STUDIO ON AUTOMOBILE

(71) Applicants: Mara Lee Chaben, Culver City, CA (US); Cooper Timothy Anstett, Nashville, TN (US)

(72) Inventors: Mara Lee Chaben, Culver City, CA (US); Cooper Timothy Anstett, Nashville, TN (US)

(21) Appl. No.: 16/420,976

(22) Filed: May 23, 2019

(51) Int. Cl. A61M 21/02; A61G 99/00; G06Q 50/10; G06Q 30/00; G06Q 10/00

(52) U.S. Cl. CPC A61M 21/02; A61G 99/00; G06Q 10/20; G06Q 30/016; G06Q 50/10

(57) ABSTRACT

Referring to the drawing, there is shown a school bus (10), which has a fully renovated, empty interior with wooden floors & heightened ceilings (12), provides a sense of serene calm (12), smells pleasant (12), and a door that does not allow personal distractions (14). The invention will now be described in use, with reference to preferred embodiment. In use, the automobile (10) arrives at a business, institution, organization, school, neighborhood, event or designated side walk. Clients sign up for one of the variety of classes prior to boarding through the main door (14). Inside the vehicle (12), they experience a guided mindfulness or yoga practice. Then clients go on with their day, reaping all the benefits of consistent group mindfulness or yoga practice without deviating from their busy schedule.

### The present invention relates to a yoga & meditation studio on an automobile including but not limited to any type of bus or large van.

### BACKGROUND

Science proves & people understand the health benefits of a regular mindfulness and yoga practice.

However, people are busy, distracted and inconsistent. Mindfulness training is ineffective unless practiced on a repetitive basis.

There is a lack of accessible, quick, live, group mindfulness and yoga practices that are easy to fit in a busy schedule.

### SUMMARY

The present invention seeks to provide a solution to this problem by providing a yoga & meditation studio on a renovated automobile that visits businesses, institutions, organizations, schools, neighborhoods and events to bridge the gap between group mindfulness or yoga practice and people's busy, daily lives.

A yoga and meditation studio on a renovated automobile provides group mindfulness classes with a variety of themes. The studio has a set class & location schedule.

In a preferred embodiment, all necessary props for class and a sound system are included.

Additionally, the renovations of the yoga and meditation studio are aesthetically impressive, calming and welcoming.

The present invention will now be more particularly described, with reference to the accompanying drawing, by way of example only and in no way limiting to the scope of the invention, in which:

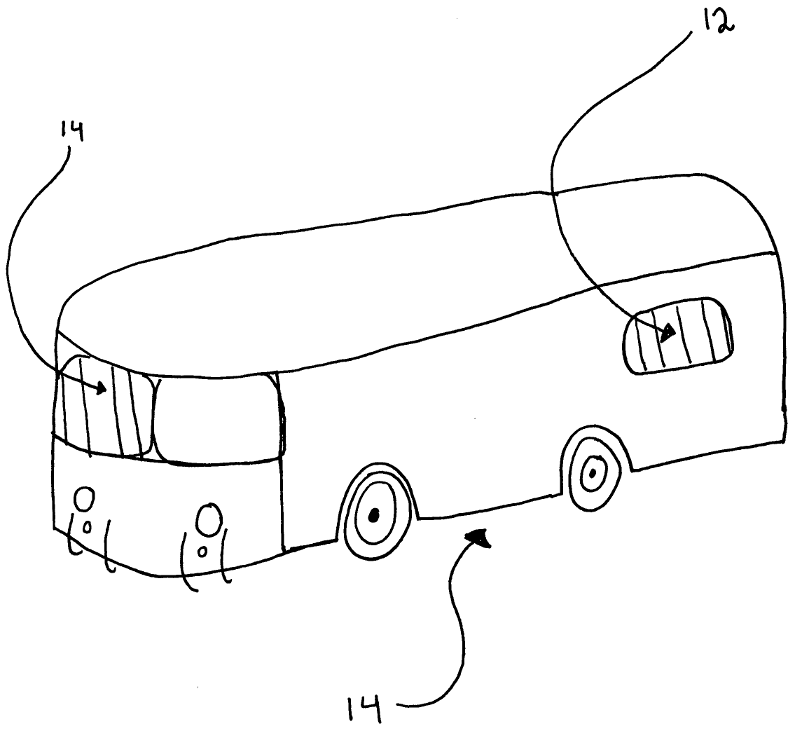

FIG. 1 is an exterior side view of a renovated school bus, which encompasses the yoga & meditation studio.

### DETAILED DESCRIPTION

Referring to the drawing, there is shown a school bus (10), which has a fully renovated, empty interior with wooden floors & heightened ceilings (12), provides a sense of serene calm (12), smells pleasant (12), and a door that does not allow personal distractions (14).

The invention will now be described in use, with reference to preferred embodiment.

In use, the automobile (10) arrives at a business, institution, organization, school, neighborhood, event or designated side walk. Clients sign up for one of the variety of classes prior to boarding through the main door (14). Inside the vehicle (12), they experience a guided mindfulness or yoga practice. Then clients go on with their day, reaping all the benefits of consistent group mindfulness or yoga practice without deviating from their busy schedule.

1. A renovated automobile turned mindfulness and yoga studio comprising:
a. an empty interior, fully renovated with wooden floors, heightened ceilings and a door that does not allow personal distractions.
b. a sense of calm and pleasant smell to ensure a serene space.

2. The automobile in use as set forth in claim 1, wherein the studio arrives at a set location (business, institution, organization, school, neighborhood, event or designated side walk), clients sign up for the classes and then inside the vehicle, the clients experience a guided mindfulness or yoga practice.
